(19) United States
(12) Patent Application Publication
(10) Pub. No.: US 2018/0153508 A1
(43) Pub. Date: Jun. 7, 2018

(54) ULTRASONIC PROBE, ULTRASONIC UNIT, AND SUBJECT INFORMATION ACQUISITION APPARATUS

(71) Applicant: CANON KABUSHIKI KAISHA, Tokyo (JP)

(72) Inventor: Atsushi Kandori, Ebina-shi (JP)

(21) Appl. No.: 15/826,516

(22) Filed: Nov. 29, 2017

(30) Foreign Application Priority Data
Dec. 7, 2016 (JP) 2016-237688

(51) Int. Cl. A61B 8/00; A61B 5/00; G01S 15/89

(52) U.S. Cl. CPC A61B 8/4444; G01S 15/8965; A61B 5/0095

(57) ABSTRACT

An ultrasonic probe includes an element configured to include a diaphragm unit for at least receiving or transmitting an ultrasonic wave and a chassis configured to extend in a direction vertical to a diaphragm plane included in the diaphragm unit and hold the element. A center of the diaphragm plane of the element in an in-plane direction is arranged to be offset from a center of the chassis.

### BACKGROUND

### Field of the Disclosure

The present disclosure relates to an ultrasonic probe performing transmission/reception (in the present specification, transmission/reception means at least either transmission or reception) of an acoustic wave such as an ultrasonic wave, an ultrasonic unit having arranged therein a plurality of ultrasonic probes, and a subject information acquisition apparatus using the ultrasonic unit.

### Description of the Related Art

In recent years, a photoacoustic imaging apparatus imaging an inside of a living body with use of a photoacoustic effect is being studied and developed. In the photoacoustic imaging apparatus, a living body is irradiated with a pulse laser beam (laser pulse) emitting for a short period, and an image is generated from an ultrasonic wave (photoacoustic wave) generated when the living tissue absorbing energy of the pulse laser beam expands in volume due to generation of heat. The photoacoustic imaging apparatus is studied and developed as an apparatus for observing a human breast for breast cancer early detection, for example. U.S. Pat. No. 5,713,356 discloses a scanner apparatus for breast observation in which, as illustrated in FIG. 22, a plurality of ultrasonic transducers 1533 are arranged in a hemispherical rotation supporting member 1552 (a rotation shaft 1558 is provided) to obtain an acoustic wave from a subject. In U.S. Pat. No. 5,713,356, a piezoelectric body is used as the ultrasonic transducer. Also, US2015/0268091A1 discloses a photoacoustic imaging apparatus in which capacitive micromachined ultrasonic transducers (CMUTs) each serving as a capacitive ultrasonic transducer are arranged in a hemispherical supporting member. The photoacoustic imaging apparatus will be described with reference to FIG. 21. A supporting member 1413 includes a hemispherical unit 1420, and a plurality of capacitive micromachined ultrasonic transducers (CMUTs) are arranged in the hemispherical unit. An optical fiber 1434 guides a laser beam from a light source. Here, the capacitive micromachined ultrasonic transducer (CMUT) is described in A. S. Ergun, Y. Huang, X. Zhuang, O. Oralkan, G. G. Yarahoglu, and B. T. Khuri-Yakub, “Capacitive micromachined ultrasonic transducers: fabrication technology,” Ultrasonics, Ferroelectrics and Frequency Control, IEEE Transactions on, vol. 52, no. 12, pp. 2242-2258, December 2005, and is fabricated by means of a micro electromechanical systems (MEMS) process, to which a semiconductor process is applied. FIG. 20 is a schematic view illustrating a cross-section of an example of a CMUT (transmission/reception element). In FIG. 20, a vibrating film 1101, and a first electrode 1102 and a second electrode 1103 opposed with a space (cavity) 1105 interposed therebetween, are referred to collectively as a cell. The vibrating film 1101 is supported by a supporting unit 1104 formed on a chip (substrate) 1100. The second electrode 1103 is connected to a direct-current (DC) voltage generation unit 1202. To the second electrode 1103, predetermined DC voltage Va is applied by the DC voltage generation unit 1202 via a second line 1302. On the other hand, the first electrode 1102 is connected to a transmission/reception circuit 1201 via a first line 1301 and is at a fixed electric potential around a ground (GND) potential. This causes a potential difference Vbias=Va−0V to be generated between the first electrode 1102 and the second electrode 1103. By adjusting a value of the Va, a value of the Vbias corresponds to a desired potential difference (approximately tens of volts to hundreds of volts) determined by mechanical properties that the cell of the CMUT has. The transmission/reception circuit 1201 applies alternating-current (AC) driving voltage to the first electrode 1102 to cause an AC electrostatic attractive force to be generated between the first and second electrodes, which enables the vibrating film 1101 to be vibrated at a certain frequency and enables an ultrasonic wave to be transmitted. Also, the vibrating film 1101 receives the ultrasonic wave and is vibrated to cause fine current to be generated in the first electrode 1102 due to electrostatic induction. The transmission/reception circuit 1201 measures a value of the current, and a reception signal can be obtained.

An efficient way to distribute and arrange a plurality of capacitive micromachined ultrasonic transducers (CMUTs) on a surface of a hemisphere serving as a supporting member to constitute an ultrasonic unit (ultrasonic probe unit) is to prepare an ultrasonic probe obtained by housing a single element and an associated circuit in a chassis in order to secure efficiency and reliability of assembly. The specific ultrasonic unit can be formed by preparing a hemispherical supporting member having a plurality of holes each corresponding to an external shape of the ultrasonic probe and inserting and securing the ultrasonic probes into the holes. The specific ultrasonic unit can also be formed by arranging and securing the plurality of ultrasonic probes on the inner surface of the hemispherical supporting member so that a diaphragm plane of elements, including diaphragm units, of the ultrasonic probes may face in a direction toward a center of the hemisphere.

In arranging the plurality of ultrasonic transducers (ultrasonic probes), when the element interval between the ultrasonic transducer elements is larger, this facilitates generation of an artifact (aliasing) at the time of reconstructing a photoacoustic imaging image (or an ultrasonic imaging image) based on signals obtained from the transducer elements. Thus, it is important to distribute and arrange the ultrasonic transducers (ultrasonic probes) as closely to each other as possible in a high-density state. However, although it is desirable to reduce the size of the ultrasonic probe for achievement of the high-density arrangement, there is a problem in which, since a region for electric connection from the chip (element substrate) provided with the ultrasonic transducer (CMUT) to the circuit is required, this is an obstacle to size reduction of the ultrasonic probe.

### SUMMARY

An object of the present disclosure is to provide an ultrasonic probe including a single-element ultrasonic transducer that can be arranged in a hemispherical supporting member in a high-density state. An ultrasonic probe according to the present disclosure includes an element configured to include a diaphragm unit for at least receiving or transmitting an ultrasonic wave and a chassis configured to extend in a direction vertical to a diaphragm plane included in the diaphragm unit and hold the element. A center of the diaphragm plane of the element in an in-plane direction is arranged to be offset from a center of the chassis.

Further features of the present disclosure will become apparent from the following description of exemplary embodiments (with reference to the attached drawings).

### BRIEF DESCRIPTION OF THE DRAWINGS

FIG. 1 is a schematic view illustrating an ultrasonic probe according to the present disclosure.

FIG. 2 is a schematic view illustrating the ultrasonic probe according to the present disclosure.

FIG. 3 is a schematic view illustrating the ultrasonic probe according to the present disclosure.

FIG. 4 is a schematic view illustrating the ultrasonic probe according to the present disclosure.

FIG. 5 is a schematic view illustrating the ultrasonic probe according to the present disclosure.

FIG. 6 is a schematic view illustrating the ultrasonic probe according to the present disclosure.

FIG. 7 is a schematic view illustrating the ultrasonic probe according to the present disclosure.

FIG. 8 illustrates an ultrasonic probe according to a second embodiment.

FIG. 9 illustrates the ultrasonic probe according to the second embodiment.

FIG. 10 illustrates an ultrasonic probe according to a third embodiment.

FIG. 11 illustrates the ultrasonic probe according to the third embodiment.

FIG. 12 illustrates an ultrasonic probe according to a fourth embodiment.

FIG. 13 illustrates an ultrasonic probe according to a fifth embodiment.

FIG. 14 illustrates the ultrasonic probe according to the fifth embodiment.

FIG. 15 illustrates an ultrasonic probe according to a sixth embodiment.

FIG. 16 illustrates an ultrasonic probe according to a seventh embodiment.

FIG. 17 illustrates the ultrasonic probe according to the seventh embodiment.

FIG. 18 illustrates a subject information acquisition apparatus according to an eighth embodiment.

FIG. 19 illustrates a subject information acquisition apparatus according to a ninth embodiment.

FIG. 20 illustrates a capacitive micromachined ultrasonic transducer (CMUT), according to the prior art.

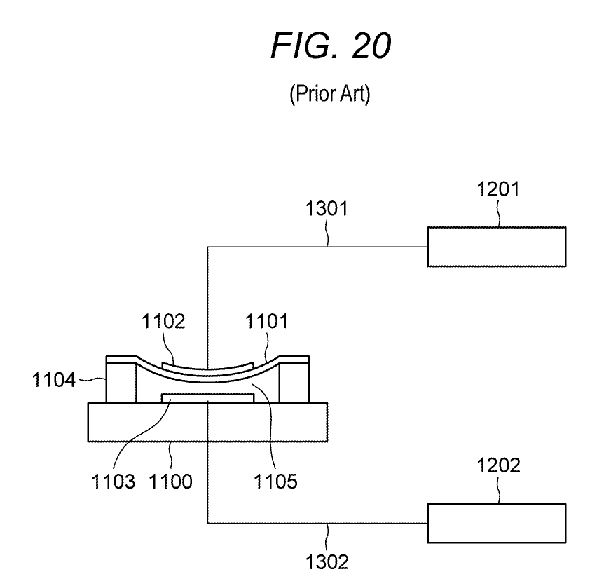

FIG. 21 illustrates a conventional photoacoustic imaging apparatus in which ultrasonic transducers are arranged in a hemispherical supporting member, according to the prior art.

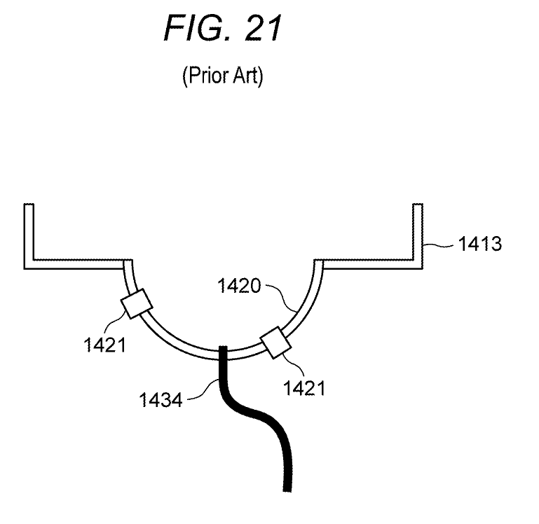

FIG. 22 illustrates a conventional photoacoustic imaging apparatus in which ultrasonic transducers are arranged in a hemispherical supporting member, according to the prior art.

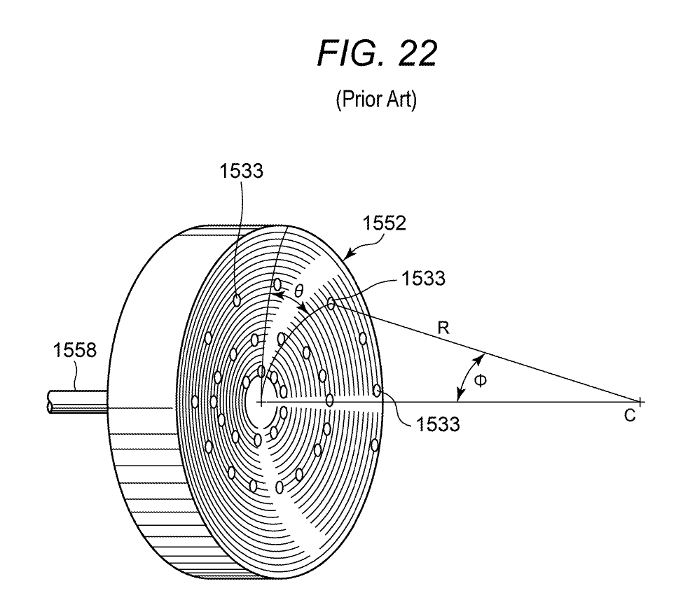

### DESCRIPTION OF THE EMBODIMENTS

To solve the above problem, the present inventor has focused attention to a positional relationship between a center of a diaphragm plane of a capacitive micromachined ultrasonic transducer in an in-plane direction and a chassis extending in a direction vertical to the diaphragm plane and holding the ultrasonic transducer. The present inventor has arrived at solving the above problem upon discovering an ultrasonic probe including an element configured to include a diaphragm unit for at least receiving or transmitting an ultrasonic wave and a chassis configured to extend in a direction vertical to a diaphragm plane included in the diaphragm unit and hold the element, wherein a center of the diaphragm plane of the element in an in-plane direction is arranged to be offset from a center of the chassis (a center of the element has an offset from a center of the chassis).

According to the present disclosure, it is possible to provide a single-element ultrasonic transducer that can be arranged in one of various supporting members such as a hemispherical supporting member including a recess in a high-density state.

In the present specification, an acoustic wave includes an elastic wave such as a photoacoustic wave, a photo-ultrasonic wave, a sound wave, and an ultrasonic wave, and an acoustic wave generated by light irradiation is particularly referred to as “a photoacoustic wave.” Also, as for the acoustic wave, an acoustic wave transmitted from a probe is referred to as “an ultrasonic wave,” and an acoustic wave into which a transmitted ultrasonic wave is reflected in a subject is referred to as “a reflected wave” in some cases. An acoustic wave is represented by an ultrasonic wave in some cases.

In the following description, as an element including a diaphragm unit for at least receiving or transmitting an ultrasonic wave, a capacitive micromachined ultrasonic transducer having a cell structure in which one of paired electrodes is provided with a vibrating film will mainly be described. However, a piezoelectric transducer using a piezoelectric element as a diaphragm unit will not be eliminated.

Hereinbelow, embodiments of the present disclosure will be described with reference to the drawings. However, the present disclosure is not limited to the embodiments and can be modified and altered in various ways within the scope thereof.

### First Embodiment

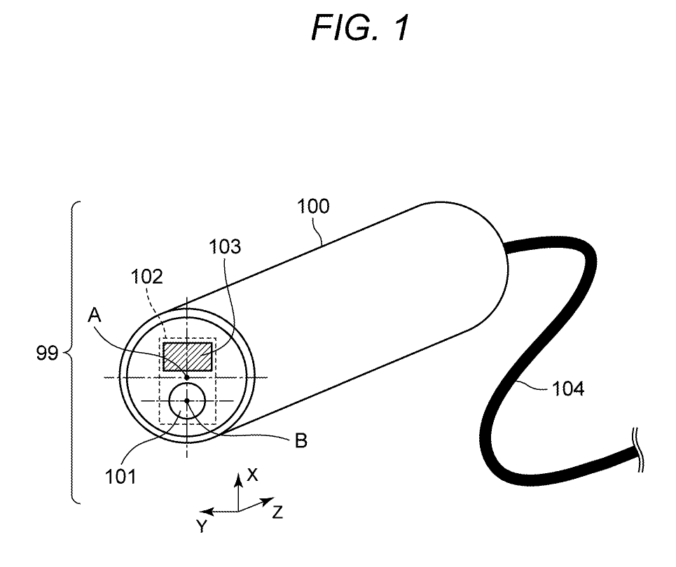

Referring to FIGS. 1 to 7, an embodiment of the present disclosure will be described. FIG. 1 is a schematic view illustrating an ultrasonic probe according to the present disclosure. In FIG. 1, an ultrasonic probe 99 includes an element 101 including a diaphragm unit for at least receiving or transmitting an ultrasonic wave. A specific example of the element is a capacitive transducer. The ultrasonic probe 99 also includes a chassis 100 extending in a direction vertical to a diaphragm plane included in the diaphragm unit of the element 101 and holding the element 101, an element substrate (chip) 102 having the element 101 mounted thereon, an electric connection unit 103 connected to the element 101, and a cable 104.

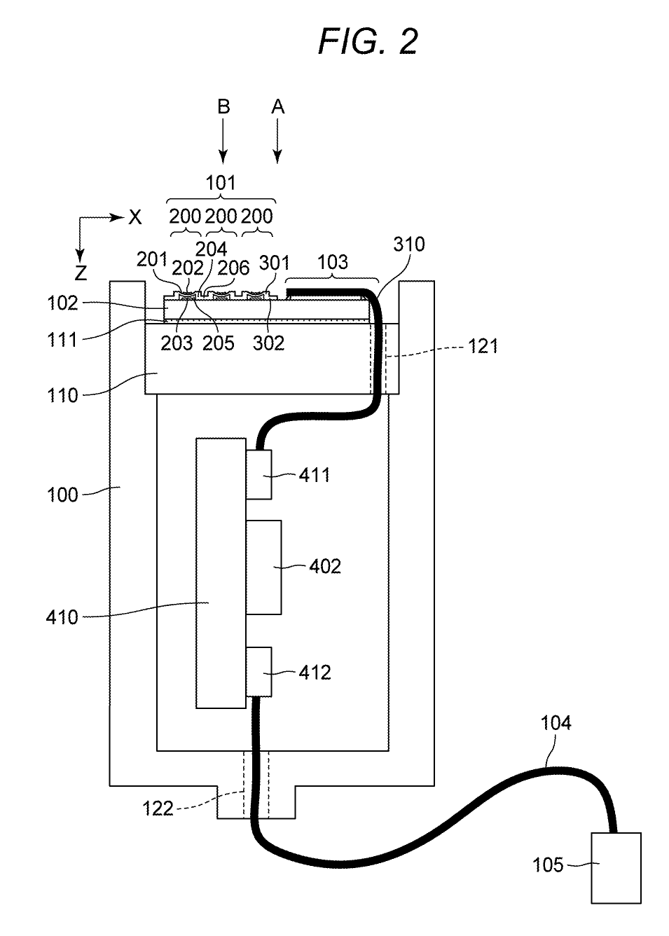

The ultrasonic probe 99 is configured to include the element 101, the circular columnar chassis 100, and the cable 104 transmitting and receiving an electric signal to and from an outside of the ultrasonic probe. From a bottom surface, which is one surface of the chassis 100, the cable 104 is extracted, and the other surface is provided with the element substrate (chip) 102 including the element 101 enabling reception or transmission/reception. As a significant characteristic of the present disclosure, as seen on the side of the other surface in FIG. 1, a center B of the element 101 is arranged to be offset (has an offset) from a center A of the chassis 100 (center of the columnar chassis in a diameter direction) and does not correspond to the center A. The center B of the element is a center of a diaphragm plane of the element in an in-plane direction and will be described below with reference to FIG. 2.

On the element substrate 102, the electric connection unit 103 configured to electrically connect the element 101 to a circuit is provided, as well as the element 101. In the configuration according to the present disclosure, since the center B of the element 101 is offset from the center A of the chassis 100, a dead space except the element substrate 102 can be minimized, and the size of the chassis 100 can be reduced. This can be interpreted as providing the electric connection unit 103 connecting the element to the circuit, which is required in addition to the element 101, to be coplanar with the element 101 (on the substrate 102) and efficiently using the dead space, in forming the ultrasonic probe. Thus, the size of the chassis 100 of the ultrasonic probe 99 can be reduced further than in a case of using the same-size substrate 102 and arranging the element 101 having no offset. That is, in a case in which the element substrate (chip) 102 having the same surface area is used, more ultrasonic probes 99 can be arranged. In other words, the elements 101 can be arranged more closely to each other in a high-density state.

Meanwhile, as for the length of the element 101 serving as a capacitive transducer, the diameter thereof is generally in a range from 0.3 mm to 1 cm, desirably in a range from 0.5 mm to 8 mm, and more desirably in a range from 1 mm to 7 mm. Here, the length is shown by the diameter. However, in a case in which the element is rectangular, the diameter in the above range can be replaced with the length of one side. Also, the size of the electric connection unit 103 in a longitudinal (longer-side) direction of the substrate (chip) 102 is generally in a range from 300 m to 1 cm, desirably in a range from 400 μm to 8 mm, and more desirably in a range from 500 m to 5 mm. In a case in which the substrate (chip) 102 is a silicon substrate, the approximate horizontal and vertical size thereof is slightly over 1 mm to 5 mm×slightly below 1 mm to several mm.

With reference to FIG. 2, an internal structure of the ultrasonic probe 99 will be described. FIG. 2 is a schematic cross-sectional view of the columnar ultrasonic probe 99 as seen in a direction of the height of the column. In FIG. 2, a supporting member 110 supporting the substrate (chip) 102 supports the substrate 102 via an adhesive 111. A through hole 121 is provided in the supporting member 110, and a through hole 122 is provided in the chassis 100. A cell 200 is configured to include a pair of electrodes, and the pair of electrodes consists of a first electrode 202 arranged on a vibrating film 201 and a second electrode 203. Also provided are a supporting unit 204 supporting the vibrating film 201, a cavity 205, and an insulation film 206. Further provided are a flexible printed wire 310, a circuit board (PCB: printed circuit board) 410, a flexible connector 411, and a cable connector 412.

Here, FIG. 2 illustrates a case in which three cells 200 are provided. The number of cells is arbitrarily selected in consideration of desired frequency characteristics and the like generally in a range from 100 to 5000, desirably in a range from 300 to 4000, and more desirably in a range from 500 to 2000. The B in FIG. 2 corresponds to the B in FIG. 1 and represents the center of the element. Here, the center B of the element represents a center of the diaphragm plane defined by the vibrating films 201 included in the plurality of cells 200 in an in-plane direction. In FIG. 2, the center B of the diaphragm plane of the element in the in-plane direction is a center of the three cells, and in a case in which the number of cells increases, the center B can be a center of the cells located on both end portions. The A in FIG. 2 represents a center of the chassis 100 (a center of the columnar chassis in a diameter direction) in a similar manner to FIG. 1.

Here, the center of the element is arranged to be offset from the center of the chassis generally in a range from 300 μm to 5 mm, desirably in a range from 500 μm to 4 mm, and more desirably in a range from 700 μm to 2 mm.

The element substrate 102 including the element 101 serving as the capacitive transducer is secured on the supporting member 110 by means of the adhesive 111. The supporting member 110 is secured to the chassis 100 to be parallel to a bottom surface of the chassis 100. A reception circuit 402 (or a transmission/reception circuit 403) is arranged on the circuit board (PCB) 410. The reception circuit 402 (or the transmission/reception circuit 403) is electrically connected to the substrate 102 via the flexible printed wire 310. The circuit board (PCB) 410 and the flexible printed wire 310 are connected via the flexible connector 411. A connector 105 for connection to an outside of the CMUT unit 99 is connected to the circuit board (PCB) 410 via a cable 104 and a cable connector 412.

Also, in the present embodiment, the circuit board 410 is formed in an elongated external shape and is arranged vertically to the element substrate 102 to face the rear side of the surface of the element substrate 102 on which the element is held. The surface area required for the circuit board 410 is larger than the area of the substrate 102. According to the present disclosure, by using the circuit board 410 formed in the elongated external shape and arranging the circuit board 410 vertically to the element substrate 102, the external shape of the CMUT unit 99 can be thin.

Meanwhile, although the positional relationship between the circuit board 410 and the substrate 102 is vertical in the above description, the positional relationship between the circuit board 410 and the substrate 102 may be approximately vertical by slightly tilting the circuit board 410 from the vertical state without an influence on the size of the external shape of the CMUT unit 99.

Although the connectors 411 and 412 are used to connect the flexible printed wire 310 to the circuit board (PCB) 410 and to connect the cable 104 to the circuit board (PCB) 410 in FIG. 2, the present disclosure is not limited to this. A configuration in which the electrode of the circuit board (PCB) 410 is directly connected to the electrode of the flexible printed wire 310 and the core line of the cable 104 is available. This configuration is more desirable than the configuration of using the connectors since the circuit board (PCB) 410 can be shorter in width, the ultrasonic probe 99 can thus be smaller in diameter, and the ultrasonic probe 99 can be arranged in a higher-density state.

Also, the chassis 100 formed in a columnar shape can be employed. An example thereof is a circular columnar chassis. Generally, the circular columnar chassis having a diameter (outside dimension) in a range from 5 mm to 15 mm can be used. Desirably, the circular columnar chassis having a diameter (outside dimension) in a range from 6 mm to 12 mm can be used. More desirably, the circular columnar chassis having a diameter (outside dimension) in a range from 7 mm to 10 mm can be used. An example in which a rectangular columnar shape is employed as the columnar shape will be described in a fourth embodiment.

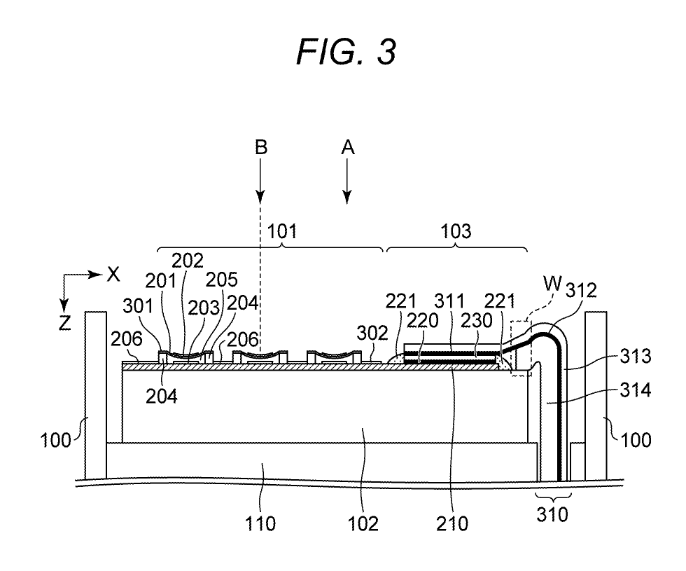

With reference to FIG. 3, the electric connection unit 103 between the substrate 102 and the flexible printed wire 310 will be described. FIG. 3 is an enlarged schematic cross-sectional view around the substrate 102 in FIG. 2. FIG. 3 shows an insulation film 210, an connection electrode 220, a flowing anisotropic insulating resin 221, an anisotropic conductive resin 230, the flexible printed wire 310, a connection electrode 311, a conductive layer 312, an insulation film 313, and an insulation film (cover lay film) 314.

The flexible printed wire 310 is configured to interpose the thin conductive layer 312 between the thin insulation layers 313 and 314 from the upper and lower sides and can be bent in a flexible manner. The conductive layer 312 is generally 10 μm to tens of μm in thickness and is made of a conductive body such as copper. Each of the insulation layers 313 and 314 is 10 μm to tens of μm in thickness and is made of a soft film such as polyimide.

At an end of the flexible printed wire 310 on a side of the substrate (chip) 102, the insulating layer 313 is arranged only on one side of the conductive layer 312, and the end is exposed as the connection electrode 311. The end of the flexible printed wire 310 is arranged to be opposed to the substrate 102, and the connection electrode 220 is arranged on the substrate 102 opposed to the connection electrode 311. The connection electrode 311 and the connection electrode 220 are electrically connected by the anisotropic conductive resin 230. The anisotropic conductive resin is one in which a plurality of conductive particles are distributed in an insulating resin. The connection electrode 311 and the connection electrode 220 are pressurized until the distance therebetween is shorter than the size of the conductive particle included in the anisotropic conductive resin, and the resin is cured. Thus, the electrodes opposed vertically are electrically connected to each other. The adjacent electrodes are insulated from each other since the distance between the adjacent electrodes is sufficiently longer than the size of the conductive particle. Also, the anisotropic conductive resin flows to a region provided with no flexible printed wire 310 (corresponding to the 221 region). Since the conductive particles exist to be distributed in the insulating resin, the resin itself has an insulating property and is electrically insulated from other regions.

Since the substrate 102 and the flexible printed wire 310 are electrically connected by the anisotropic conductive resin, the height of the electric connection unit to the subject side can approximately be the height of the flexible printed wire 310. Thus, an influence of the electric connection unit on the capacitive transducer element can be as small as possible.

The size of the connection electrode 220 is 100 μm to hundreds of μm×hundreds of μm to several mm. Also, as described above, since the anisotropic conductive resin flows to the region provided with no flexible printed wire 310 in a range from hundreds of μm to several mm, the equivalent or longer distance is required to be provided from the CMUT element. On the other hand, to prevent contact and electric connection between the cross-section of the substrate 102 and the conductive layer 312 (311) of the flexible printed wire 310, the insulating layer 314 of the flexible printed wire 310 on the substrate side is arranged to overlap with the substrate 102. Thus, the external shape of the substrate 102 is set to be slightly extended so that the insulating layer 314 may reliably overlap with the substrate. In general, a dimension of 100 μm to hundreds of μm is required to be secured.

The length of the electric connection unit 103 is required to be 500 μm to 5 mm in the longitudinal (longer-side) direction of the substrate 102. This length is similar to the aforementioned length of the CMUT element 101, which is 0.5 mm to several mm. Thus, the electric connection unit 103 is equal to or larger than the CMUT element 101 in size, and the size ratio can sufficiently provide the effect of the configuration according to the present disclosure.

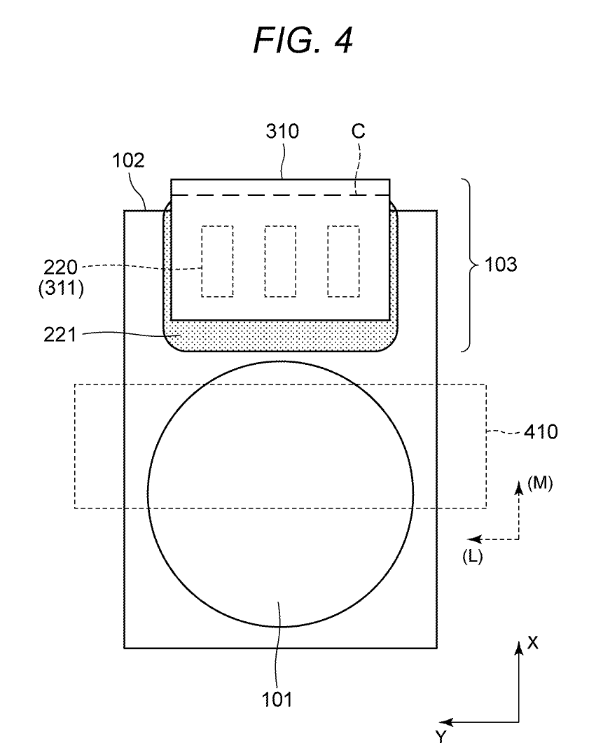

Referring to FIG. 4, arrangement on the substrate (chip) will be described. FIG. 4 is a schematic view of the substrate 102 seen from the upper side. FIG. 4 shows a folding line C of the flexible printed wire 310. The capacitive transducer element 101 is arranged on the lower side of the substrate 102 in the figure while the electric connection unit 103 for connection to the circuit 402 (403) is arranged on the upper side of the substrate 102 in the figure. As described with reference to FIG. 3, in the electric connection unit 103, the substrate and the end of the flexible printed wire 310 are opposed to each other and are electrically connected by the anisotropic conductive resin. In FIG. 4, three connection electrodes 220 formed in shapes elongated in the longitudinal (longer-side) direction of the substrate 102 (X-direction) are arranged on the substrate 102, for example. The three connection electrodes 220 are connected to the first electrode of the element 101, the second electrode of the element 101, and an electrode for holding potential of the chip 102, respectively.

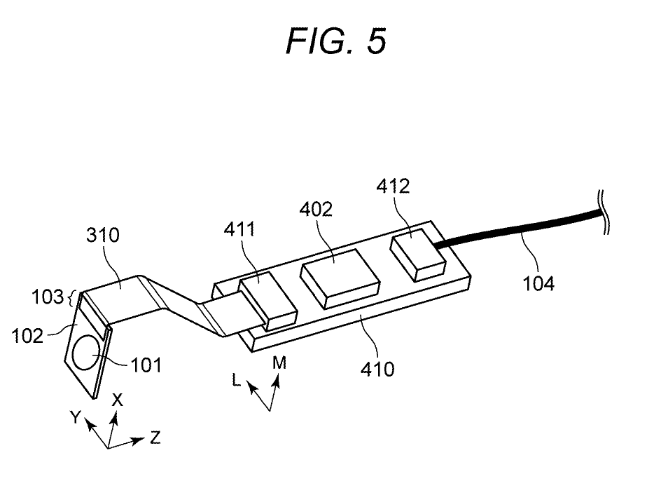

Referring to FIG. 5, the positional relationship between the substrate 102 and the circuit board 410 will be described. FIG. 5 is a schematic view illustrating only the substrate 102, the flexible printed wire 310, and the circuit board 410.

The flexible printed wire 310 extracted from the wire of the substrate 102 is extracted approximately orthogonally to the substrate 102, and the circuit board 410 is also extracted approximately orthogonally to the substrate 102. The substrate 102 and the circuit board (PCB) 410 are arranged so that the longitudinal (longer-side) direction of the substrate 102 (X-direction) and the width direction of the circuit board 410 (L-direction) may approximately be vertical to each other when the ultrasonic probe 99 is seen in a direction of the bottom surface. FIG. 5 illustrates a schematic view to which the positional relationship between the substrate 102 and the circuit board 410 is projected. Since the flexible printed wire 310 is extracted straightforwardly, the length of the wire 310 can be minimum. Thus, parasitic capacitance in the wire between the element 101 and the reception circuit 402 (transmission/reception circuit 403) can be restricted to the minimum. An influence of the parasitic capacitance on a transmission characteristic (or a reception characteristic) of the capacitive transducer can be small, and the capacitive ultrasonic probe excellent in reception (transmission/reception) characteristic can be provided.

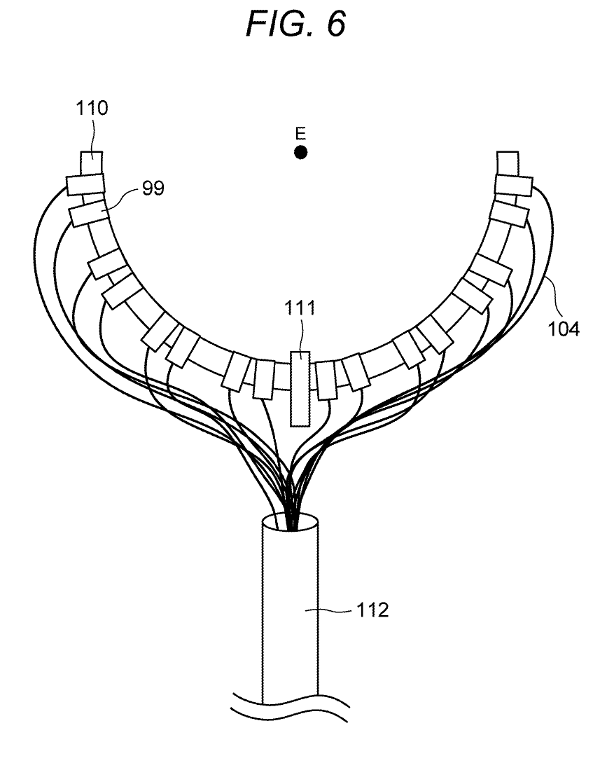

Referring to FIG. 6, an ultrasonic unit configured by arranging the plurality of capacitive transducers 99 in the hemispherical (bowl-shaped) supporting member 110 including recesses will be described. FIG. 6 shows a light source (light irradiation unit) 111 and a line 112 binding up cables. The supporting member 110 is provided with a plurality of through holes each corresponding to the external shape and the outside diameter of the ultrasonic probe 99, and the element constituting the ultrasonic probe is inserted and secured into each of the through holes to face a center E of the hemisphere.

As a subject in the present disclosure, a breast, a hand, a foot, and another part of a living body, a non-living body material, and the like can be assumed. For example, when an apparatus that can measure the breast as a subject is assumed, the radius of the hemisphere of the hemispherical chassis 110 can be in a range from 100 mm to 150 mm. The radius of the hemisphere is more desirably in a range from 110 mm to 130 mm.

In the present embodiment, the center of the capacitive reception element (transmission/reception element) is offset from the center of the chassis, and the size of the offset is most desirably about a half of the size of the electric connection unit. This enables the ultrasonic probe to be formed with respect to the chip 102 and the electric connection unit 103 most efficiently (smallest). Thus, the specific size of the offset is generally hundreds of μm to several mm.

As described above, according to the present disclosure, the center of the capacitive transducer element can be offset from the center of the chassis, and it is possible to provide the ultrasonic unit in which the plurality of ultrasonic probes each including the single element are arranged in the hemispherical supporting member including the recesses in a high-density state.

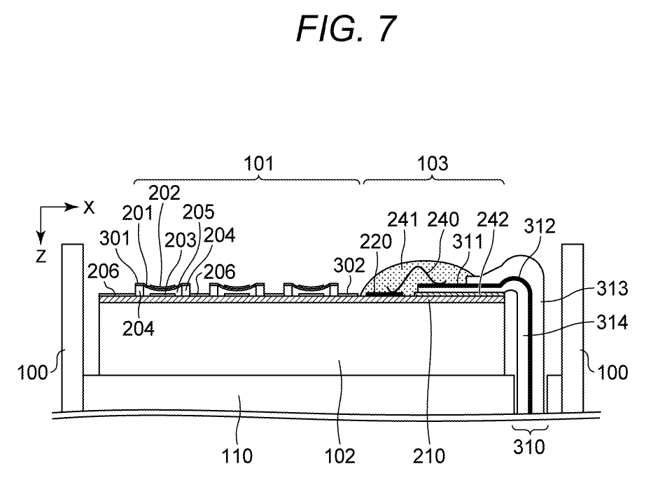

Meanwhile, although the electric connection unit 103 is configured to cause the substrate (chip) and the end of the flexible printed wire 310 to be opposed to each other and electrically connected to each other by means of the anisotropic conductive resin in the above description, the present disclosure is not limited to this configuration. Available are a configuration in which the substrate (chip) and the end of the flexible printed wire 310 are opposed to each other and electrically connected to each other in another way and a configuration in which the chip and the end of the flexible printed wire 310 are arranged in parallel and in which a wire bonder is used as illustrated in FIG. 7. FIG. 7 shows a wire 240, a sealing material 241, and an adhesive 242.

Meanwhile, although the three connection electrodes 220 are provided on the substrate 102 in the above description, the present disclosure is not limited to this configuration. The connection electrodes 220 can be used in a similar manner even in a case in which the number of the connection electrodes 220 is two to cause the electrodes to be connected to the first and second electrodes or another number, and even in a case in which the shape of each connection electrode 220 differs.

Meanwhile, in the present embodiment, the external shape of the chassis constituting the ultrasonic probe 99 is circular, and the center of the element 101 is offset from the center of the chassis. Accordingly, by securing the ultrasonic probe 99 to the supporting member including the through holes while adjusting the orientation in consideration of the offset direction, the element 101 can be arranged at a predetermined coordinate position. Also, the positional adjustment of the ultrasonic probe 99 can be performed easily by providing the chassis 100 with a pin, a protrusion, or the like for the positional adjustment.

Meanwhile, in the present embodiment, although nothing is filled between the substrate 102 and the chassis 100 in the above description, the present disclosure is not limited to this configuration. A resin which is insulated and which hardly influences the characteristics of the CMUT element, such as a silicon resin, can be filled. This brings about effects such as inter-line insulation in the CMUT element, improved insulation from an external subject, and prevention of breakage of the CMUT element caused by contact.

### Second Embodiment

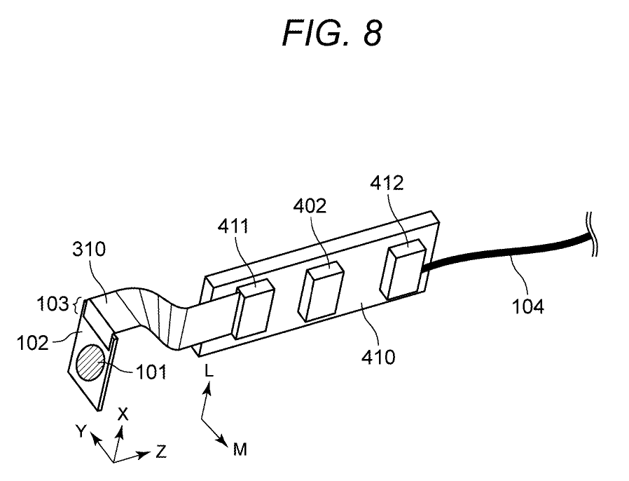

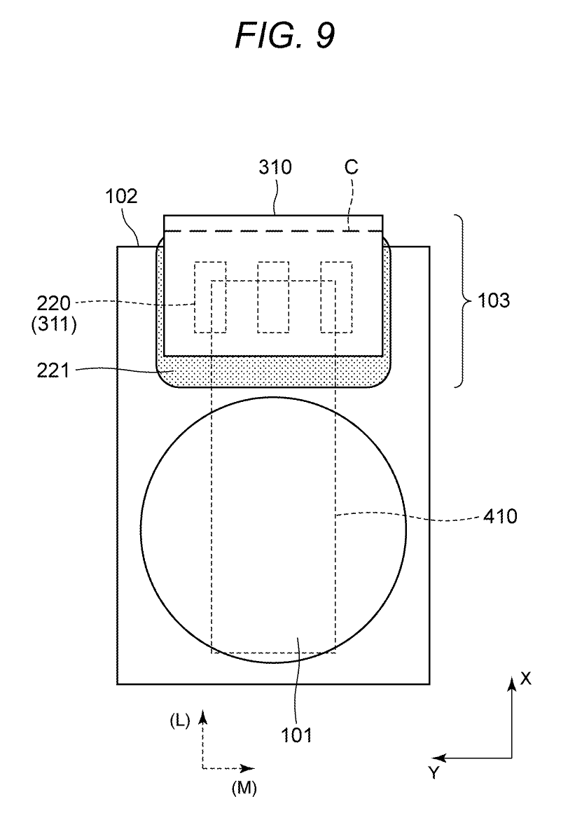

A second embodiment differs from the first embodiment in terms of the positional relationship between the element substrate (chip) 102 and the circuit board 410. The second embodiment is similar to the first embodiment in the other respects. The second embodiment will be described with reference to FIGS. 8 and 9. FIG. 8 is a schematic perspective view illustrating the positional relationship between the element substrate 102 and the circuit board 410. FIG. 9 is a schematic projection view illustrating the positional relationship between the element substrate 102 and the circuit board 410.

The second embodiment differs from the first embodiment in that the orientation of the circuit board 410 as seen from the element substrate 102 is rotated by approximately 90 degrees. Specifically, the flexible printed wire 310 electrically connected to the element substrate 102 by the electric connection unit 103 is twisted by ¼ (90 degrees) rotation before reaching the circuit board 410. Accordingly, as illustrated in FIG. 9, the longitudinal (longer-side) direction (X-direction) of the chip 102 and the width direction (L-direction) of the circuit board 410 correspond. The circuit board 410 is generally approximately 0.6 mm to several mm in the thickness direction (M-direction) and is typically 1.6 mm in thickness. Also, the circuit board 410 is required to be at least several mm to 1 cm in the width direction (L-direction) in consideration of the package size of the reception (transmission/reception) circuit and the required extra length to the board ends.

In the configuration in the present embodiment in which the longitudinal (longer-side) direction (X-direction) of the element substrate 102 and the width direction (L-direction) of the circuit board 410 correspond, the respective longitudinal (longer-side) directions correspond. Accordingly, the external size of the ultrasonic probe 99 can be restricted to the minimum.

Meanwhile, in the present embodiment, although the orientation of the circuit board 410 as seen from the element substrate 102 is rotated by 90 degrees in the above description, the present embodiment is not limited to this configuration and can be applied to a case of another angle. For example, the present embodiment can be applied to a case in which the orientation of the circuit board 410 approximately corresponds to a diagonal line of the element substrate 102. In this case, in a configuration in which the thickness (M-direction) of the circuit board 410 is shortened, the external shape of the ultrasonic probe can further be small-sized.

### Third Embodiment

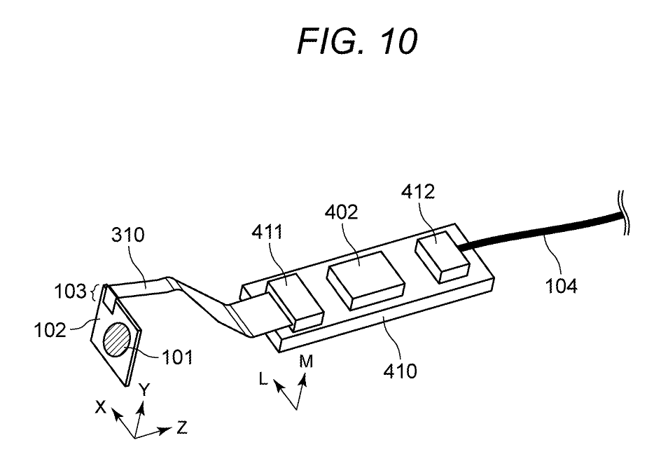

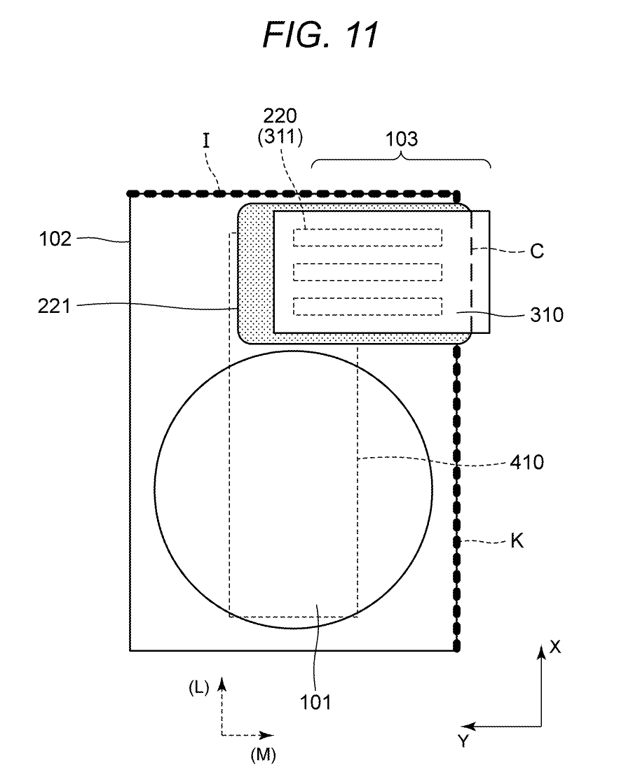

In a third embodiment, a side of the flexible printed wire 310 extracted from the element substrate (chip) 102 differs. The third embodiment is similar to the first embodiment in the other respects. The third embodiment will be described with reference to FIGS. 10 and 11. FIG. 10 is a schematic perspective view illustrating the positional relationship between the element substrate 102 and the circuit board 410. FIG. 11 is a schematic projection view illustrating the positional relationship between the element substrate 102 and the circuit board 410.

In the first embodiment, the flexible printed wire 310 is extracted from one side (side I in FIG. 11) of the element substrate 102 in a shorter-side direction. The third embodiment is characterized in that the flexible printed wire 310 is extracted from a part of one side (side K in FIG. 11) of the element substrate in a longer-side direction.

Also, since the space to fold the flexible printed wire 310 is provided at a side close to the shorter side of the element substrate 102, the wire can be extracted to the reception circuit (transmission/reception circuit) without protruding the electric connection unit 103 in the longitudinal (longer-side) direction (X-direction) of the element substrate 102. Thus, since the element substrate 102 is not required to be extended in the longitudinal (longer-side) direction (X-direction) thereof, the external shape of the ultrasonic probe 99 can be small-sized.

The present embodiment will be described from another aspect. The present embodiment is characterized in that the center of the capacitive transducer element 101 is offset from the center of the external shape of the ultrasonic probe 99 not only in the longitudinal (longer-side) direction (X-direction) of the element substrate 102 but also in the shorter-side direction (Y-direction) of the element substrate 102. Accordingly, it is possible to provide the ultrasonic unit in which the plurality of capacitive transducers each including the single element are arranged in the hemispherical supporting member in a higher-density state.

Further, since the space to cause the insulating layer (cover lay film) 314 of the flexible printed wire 310 on the side of the element substrate 102 to overlap with the element substrate 102 (region W in FIG. 3) is dispensed with, the element substrate 102 can be shortened in the longitudinal (longer-side) direction (X-direction).

Also, in the present embodiment, since the longitudinal (longer-side) direction (X-direction) of the element substrate 102 and the width direction (L-direction) of the circuit board 410 can approximately correspond even without twisting the flexible printed wire 310, the ultrasonic probe 99 can be small-sized. In the present embodiment, since the flexible printed wire 310 is not required to be twisted as in the second embodiment, the entire length of the ultrasonic probe 99 can further be shortened.

In addition, as illustrated in FIG. 11, the connection electrodes 220 on the element substrate 102 are arranged to extend in the shorter-side direction (Y-direction) of the chip. In a case in which the connection electrodes 220 on the element substrate 102 and the flexible printed wire 310 are connected by the anisotropic conductive resin, the anisotropic conductive resin tends to flow far in the longitudinal (longer-side) direction of the connection electrodes. In the configurations of the first and second embodiments, since the connection electrodes 220 are oriented in a direction in which the anisotropic conductive resin easily flows on the element substrate 102 from the electric connection unit toward the element 101, the flexible printed wire 310 is required to have a predetermined distance from the element 101. Conversely, in the third embodiment, the direction in which the anisotropic conductive resin flows from the flexible printed wire 310 can be different from the direction of the element 101. Accordingly, the distance between the flexible printed wire 310 and the element 101 can be shorter than that in the configuration according to the first embodiment, and the region of the electric connection unit 103 itself can be smaller.

In this manner, in the configuration according to the present embodiment, since the space to fold the flexible printed wire 310 can be small-sized, and enlargement of the region of the electric connection unit 103 due to flowing of the anisotropic conductive resin can be prevented, it is possible to provide the ultrasonic probes arranged in a higher-density state.

### Fourth Embodiment

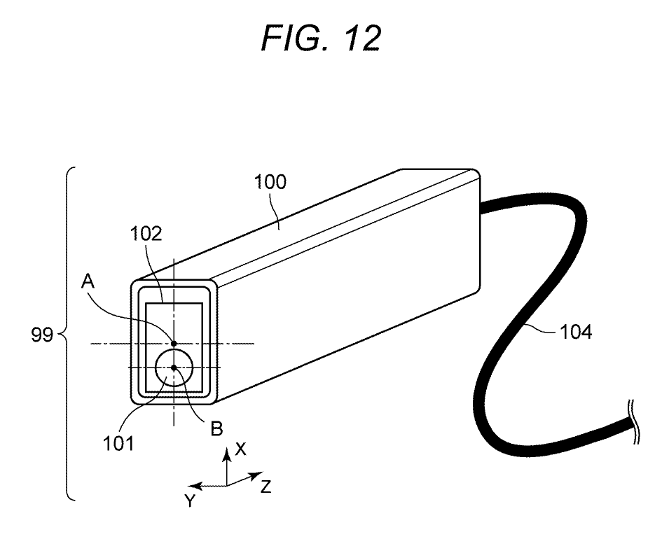

In a fourth embodiment, the external shape of the ultrasonic probe 99 differs. The fourth embodiment is similar to any of the first to third embodiments in the other respects. The fourth embodiment will be described with reference to FIG. 12. FIG. 12 is a schematic perspective view illustrating the ultrasonic probe 99.

The fourth embodiment differs in that the external shape of the ultrasonic probe 99 is quadrangular. Since the element substrate 102 is cut from a large silicon wafer, the shape of the element substrate 102 is generally quadrangular in terms of the manufacturing efficiency. In the present embodiment, since the chassis 100 is in a rectangular columnar shape, and the external shape of the chassis 100 is quadrangular as with the external shape of the chip 102, a wasted space inside the chassis 100 is hardly generated, and the external shape of the ultrasonic probe 99 can further be small-sized.

The present embodiment can exert a particularly high effect when the present embodiment is combined with the third embodiment. The combination is desirable since the ultrasonic probe 99 having the external size of the element substrate 102 slightly extended can be formed although it depends on the sizes of the element substrate 102 and the circuit board 410.

Meanwhile, in the present embodiment, although the external shape of the ultrasonic probe 99 is quadrangular in the above description, the present disclosure is not limited to this. The ultrasonic probe can be formed with use of a chassis formed in another shape such as a polygonal shape, a truncated polygonal shape, an elliptic shape, and an arbitrary shape.

### Fifth Embodiment

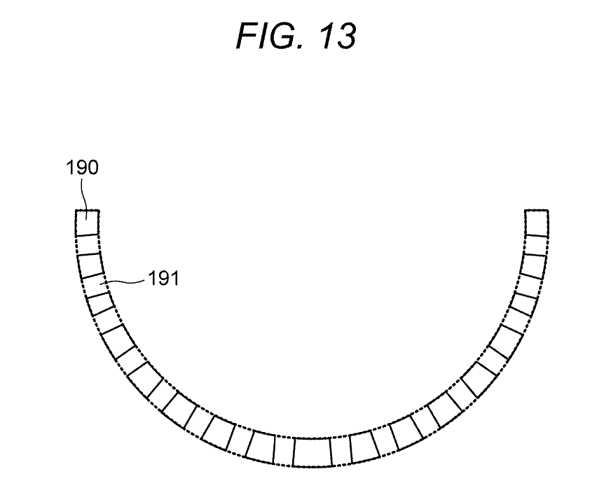

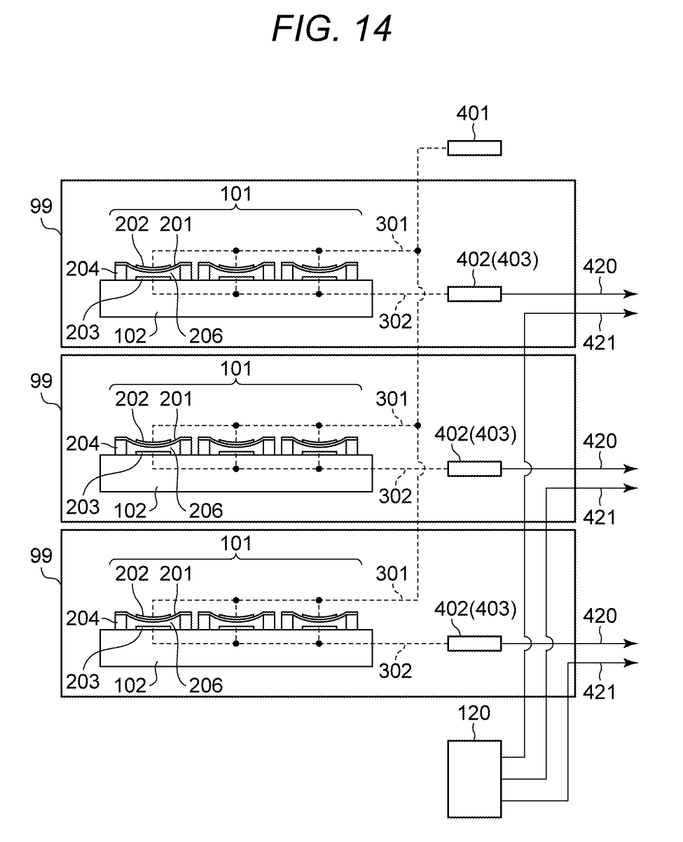

A fifth embodiment relates to a supporting member supporting the ultrasonic probes. The fifth embodiment is similar to any of the first to fourth embodiments in the other respects. The fifth embodiment will be described with reference to FIGS. 13 and 14.

As illustrated in FIG. 13, a supporting member 190 supporting the ultrasonic probes is provided with a plurality of through holes 191 each having the same size as that of the external shape of each ultrasonic probe. The ultrasonic probes are inserted in the respective through holes 191 and are mechanically secured. The fifth embodiment is characterized in that the through holes 191 opened in the supporting member 190 are arranged so that the intervals may be approximately uniform. In the present embodiment, the through holes 191 provided in the supporting member 190 are arranged uniformly. In other words, the thicknesses of remaining members between the through holes 191 of the supporting member 190 are uniform, and the supporting member 190 has no part with low strength and can have entirely uniform strength. In a case in which the ultrasonic probes are arranged in a high-density state, the intervals between the adjacent through holes 191 are short. In this case, since the supporting member tends to decrease in strength and is required to support an increasing number of probes, the supporting member is required to have high strength. In the present embodiment, since the supporting member 190 has high strength, the capacitive transducer element can receive an accurate photoacoustic signal (or an ultrasonic signal) without the coordinates thereof shifted, and subject information or an image with high quality can be acquired.

Also, in the present embodiment, since the through holes 191 provided in the supporting member 190 are uniformly arranged, and the center of each element 101 is offset from the center of the external shape of each ultrasonic probe 99 inserted and secured in each through hole, a coordinate recording unit 120 having recorded therein all coordinate information is provided as illustrated in FIG. 14. An image generation unit for the subject receives not only a reception signal 420 of each capacitive transducer element but also element coordination information 421 from the coordinate recording unit 120. The image generation unit can reconstruct an image with use of the reception signals 420 of the respective elements detected by the reception circuits 402 (or the transmission/reception circuits 403) by correcting information about the element positions. Accordingly, information about the subject can be acquired accurately due to the configuration in which the supporting member is provided with the through holes uniformly.

As described above, in the present embodiment, the through holes 191 provided in the supporting member 190 are uniformly arranged, the coordinate recording unit 120 having recorded therein coordinates of the respective elements is provided, and the element coordinate information 421 as well as the reception signals can be output as correction information. Accordingly, the ultrasonic unit enabling accurate subject information to be acquired can be provided.

Meanwhile, in the present embodiment, the configuration in which the ultrasonic probes have one coordinate recording unit 120 has been described. However, in the present disclosure, a configuration in which each ultrasonic probe 99 has the coordinate recording unit 120 is available. Also, the present disclosure is not limited to this configuration, and a configuration in which a subject information acquisition apparatus connected to the ultrasonic probes has the coordinate recording unit 120 is available.

### Sixth Embodiment

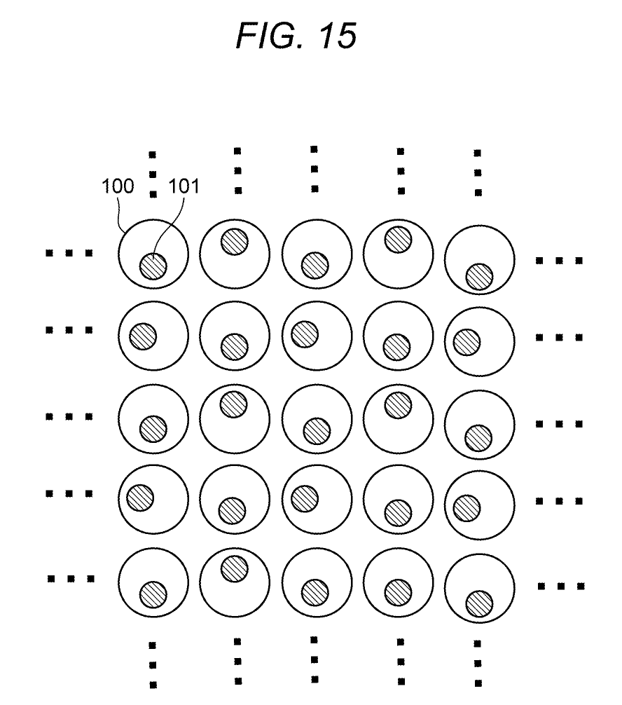

A sixth embodiment relates to the orientation of the offset of the element 101 arranged in the chassis 100 constituting each capacitive transducer probe. The sixth embodiment is similar to any of the first to fifth embodiments in the other respects. The sixth embodiment will be described with reference to FIG. 15. FIG. 15 is a schematic view of partially enlarged ultrasonic probes to describe the positional relationship between the chassis 100 and the CMUT element 101.

The sixth embodiment is characterized in that the orientation in which the center of the CMUT element 101 is offset from the center of the chassis 100 differs per probe. In a case in which the intervals of the CMUT elements 101 are uniform, artifacts (aliasing) caused by the intervals between the CMUT element 101 are easily generated. In the present embodiment, since the intervals between the CMUT elements 101 vary, the artifacts (aliasing) generated from the respective elements are not equal to each other, and as a whole, the artifacts (aliasing) can be reduced.

According to the present embodiment, it is possible to provide the ultrasonic unit in which the CMUT probes each including the single element are arranged in the hemispherical supporting member in a high-density state and in which generation of the artifacts is reduced.

### Seventh Embodiment

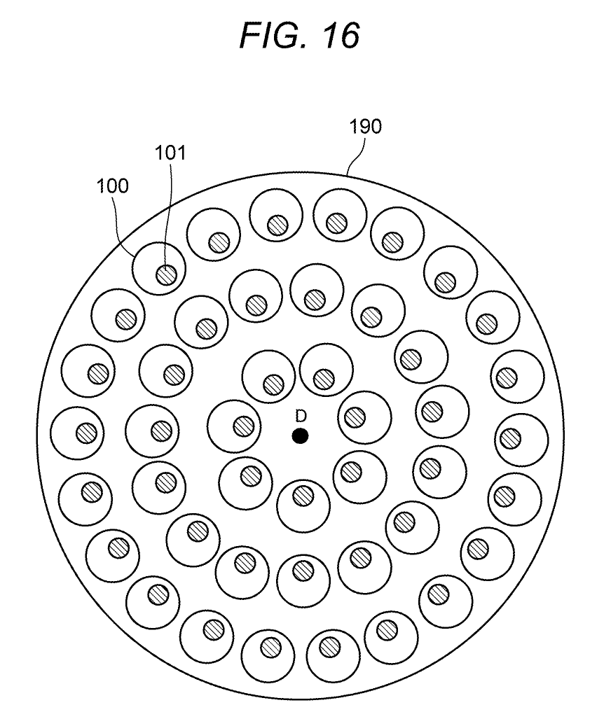

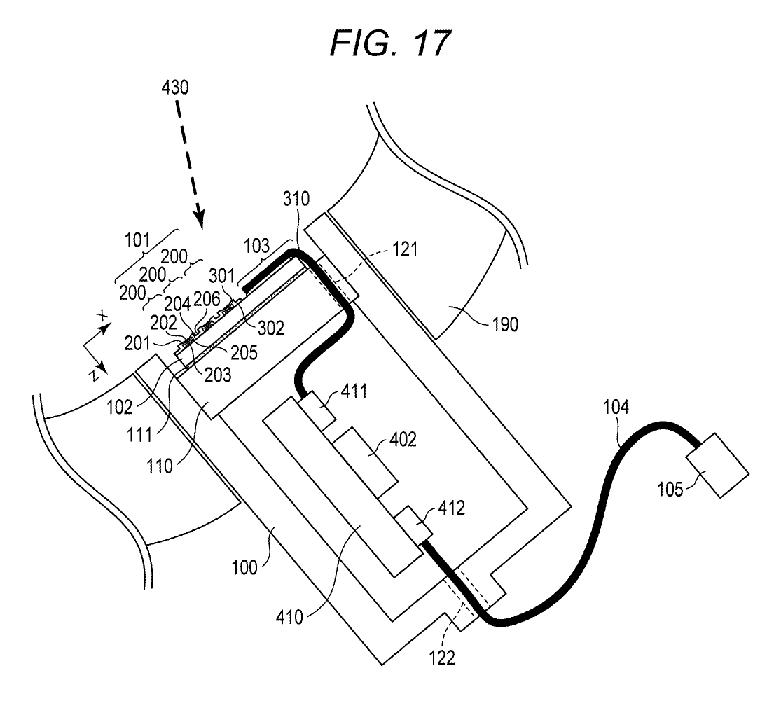

A seventh embodiment relates to the orientation of the offset of the CMUT element 101 arranged in the chassis 100 constituting each capacitive transducer probe. The seventh embodiment is similar to any of the first to fifth embodiments in the other respects. The seventh embodiment will be described with reference to FIGS. 16 and 17.

FIG. 16 is a plan view of the ultrasonic probes as seen from the subject side. As illustrated in FIG. 16, the seventh embodiment is characterized in that the CMUT elements 101 arranged in the chassis 100 each constituting the transducer probe are arranged to be offset in directions opposite from the subject at the time of being secured in the hemispherical (bowl-shaped) supporting member 190. The hemispherical bowl is in a more tapered (that is, the element intervals are decreased) structure in the direction opposite from the subject (that is, in the depth direction of the bowl, and in a direction toward the center and the back of the circle on the drawing sheet in FIG. 16). Thus, when the CMUT elements 101 are arranged at the same intervals, the number of CMUT elements that can be arranged circularly at each depth decreases in the depth direction of the bowl. Hence, the transducer probes are required to be smaller in size in the depth direction of the bowl. In the present embodiment, since the center of each CMUT element 101 is offset from the center of each chassis 100 in the depth direction of the bowl, the intervals of the adjacent CMUT elements 101 can be shorter.

Also, in the present embodiment, the center of each CMUT element 101 is offset from the center of the external shape of each chassis 100 in the direction opposite from the subject as illustrated in the schematic cross-sectional view in FIG. 17. The ultrasonic probe sometimes has a configuration in which the chassis 100 surrounding the element substrate 102 protrudes to the subject side further than the element substrate 102. A photoacoustic wave 430 from the subject reaches the CMUT element 101 from a position around the center of the bowl-shaped supporting member. In the configuration according to the present embodiment, the center of the CMUT element 101 is offset in the direction opposite from the subject. Accordingly, even in the configuration in which the chassis 100 protrudes to the subject side further than the element substrate 102, the photoacoustic signal 430 is scattered by the protrusion, and the photoacoustic signal reaching the CMUT element 101 is hardly influenced.

### Eighth Embodiment

The ultrasonic transducer described in any of the first to seventh embodiments can be used for reception of a photoacoustic wave (ultrasonic wave) with use of a photoacoustic effect and can be applied to a subject information acquisition apparatus including the ultrasonic transducer.

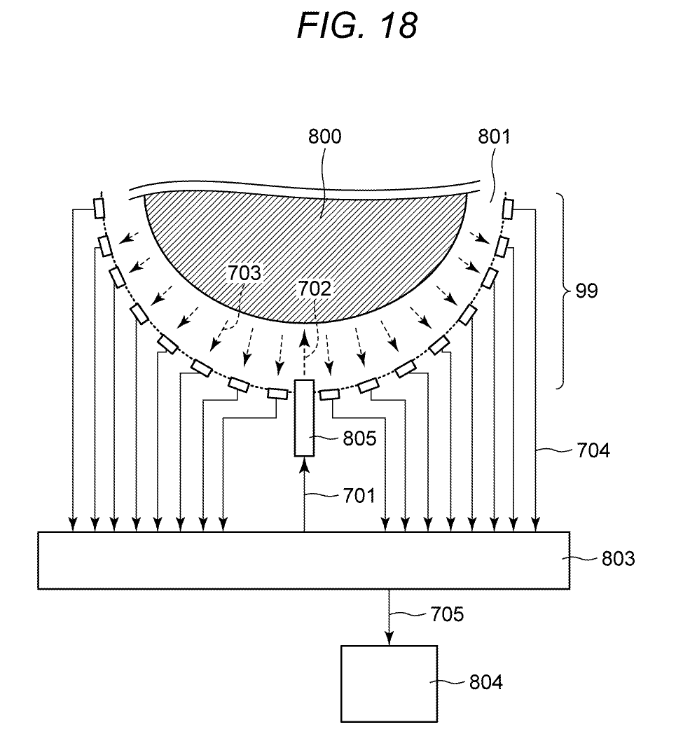

FIG. 18 is a schematic view illustrating a subject information acquisition apparatus according to the present embodiment. FIG. 18 shows a subject 800, a medium 801, the CMUT probes 99, an image information generation unit (signal processing unit) 803, an image display 804, a light source unit 805 emitting light 702, a light emission instruction signal 701, an acoustic wave (ultrasonic wave) 703 generated by emission of the light 702, a photoacoustic wave reception signal 704, and reconstructed image information 705 generated by the photoacoustic signal.

Based on the light emission instruction signal 701, the light 702 (pulse light) is generated from the light source 805 to cause the subject 800 to be irradiated with the light 702. In the subject to be measured 800, the acoustic wave (ultrasonic wave) 703 is generated by emission of the light 702 and is received by the plurality of CMUT probes 99. Between the subject information acquisition apparatus and the subject 800, the medium 801 is filled to avoid attenuation of the acoustic wave (ultrasonic wave) due to bubbles. Information about the reception signal in terms of the magnitude, shape, and time is transmitted as the photoacoustic wave reception signal 704 to the image information generation unit 803 serving as a signal processing unit. Also, information about the light 702 generated by the light source 805 in terms of the magnitude, shape, and time (light emission information) is stored in the photoacoustic signal image information generation unit 803. The photoacoustic signal image information generation unit 803 generates an image signal of the subject 800 based on the photoacoustic wave reception signal 704 and the light emission information and outputs the reconstructed image information 705 in the form of the photoacoustic signal. The image display 804 displays an image of the subject 800 based on the reconstructed image information 705 in the form of the photoacoustic signal.

According to the present embodiment, since the ultrasonic probes according to the present disclosure enable the CMUT elements to be arranged in a high-density state, it is possible to provide the subject information acquisition apparatus that can restrict generation of the artifacts (aliasing) and that can generate high-quality subject information.

### Ninth Embodiment

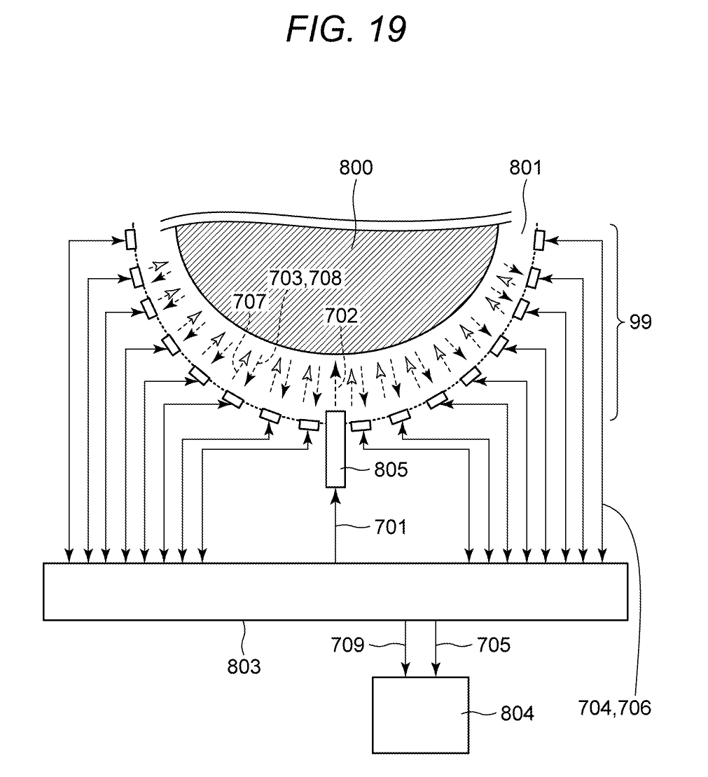

The ultrasonic transducer (CMUT) described in any of the first to seventh embodiments can be used not only for photoacoustic imaging described in the eighth embodiment but also for ultrasonic imaging with use of transmission/reception of an ultrasonic wave and can be applied to a subject information acquisition apparatus including the ultrasonic transducer. In this case, as illustrated in FIG. 19, the transmission/reception circuit 403 having a function of giving a transmission signal to the CMUT in addition to a reception function is required to be used instead of the reception circuit 402.

FIG. 19 shows a photoacoustic wave and ultrasonic wave reception signal 704, ultrasonic wave transmission information 706, a transmitted ultrasonic wave 707, a reflected ultrasonic wave 708, and reconstructed image information 709 for ultrasonic imaging.

The CMUT probes 99 have a configuration in which the plurality of CMUT elements serving as transmission/reception elements are arranged in an array. The ultrasonic wave 707 output from the CMUT probe 99 toward the subject 800 is reflected on the surface of the subject 800 due to the difference in specific acoustic impedance at the interface. The reflected ultrasonic wave 708 is received in the CMUT probe 99, and information about the reception signal in terms of the magnitude, shape, and time is transmitted as the ultrasonic wave reception signal 706 to the image information generation unit 803. Also, information about the transmitted ultrasonic wave applied to the CMUT probe 99 in terms of the magnitude, shape, and time is transmitted to the image information generation unit 803. The image information generation unit 803 generates an image signal of the subject 800 based on the ultrasonic wave reception signal 704 and the ultrasonic wave transmission information and transmits the image signal as the reconstructed image information 709, and the reconstructed image information 709 is displayed on the image display 804.

According to the present embodiment, since the ultrasonic probes according to the present disclosure enable the CMUT elements to be arranged in a high-density state, it is possible to provide the subject information acquisition apparatus that can restrict generation of the artifacts (aliasing) and that can generate high-quality subject information. Also, when the ultrasonic probes according to the present embodiment are used, different subject information can be acquired by the photoacoustic imaging and the ultrasonic imaging. Accordingly, more detailed subject information can be acquired, and a subject image having a large amount of information can be generated. Further, since reception of the photoacoustic wave and transmission/reception of the ultrasonic wave are performed with use of the same ultrasonic transducers, there is almost no misalignment between coordinates in subject information acquired by reception of the photoacoustic wave and coordinates in subject information acquired by transmission/reception of the ultrasonic wave. Hence, when the respective subject images overlap with each other, an image with little misalignment can be displayed. Meanwhile, in the embodiments in the present specification, although description is provided by connecting a DC voltage generation unit 401 to the first electrode 202 and connecting the reception circuit 402 to the second electrode 203, the present disclosure is not limited to these embodiments. The present disclosure can similarly be used for a configuration of connecting the reception circuit 402 to the first electrode 202 and connecting the DC voltage generation unit 401 to the second electrode 203.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2016-237688, filed Dec. 7, 2016, which is hereby incorporated by reference herein in its entirety.

What is claimed is:

1. An ultrasonic probe comprising:
an element configured to include a diaphragm unit for at least receiving or transmitting an ultrasonic wave; and
a chassis configured to extend in a direction vertical to a diaphragm plane included in the diaphragm unit and hold the element,
wherein a center of the diaphragm plane of the element in an in-plane direction is arranged to be offset from a center of the chassis.

2. The ultrasonic probe according to claim 1, wherein the element is a capacitive transducer having a structure of at least one cell in which one of paired electrodes is provided with a vibrating film.

3. The ultrasonic probe according to claim 2, wherein the capacitive transducer is configured to include the plurality of cells.

4. The ultrasonic probe according to claim 1, wherein the element is a transducer using a piezoelectric element.

5. The ultrasonic probe according to claim 1, comprising:
an element substrate configured to hold an electric connection unit connected to the element and the element in a direction parallel to the diaphragm plane.

6. The ultrasonic probe according to claim 5, wherein the electric connection unit is an electric connection unit connected to a flexible printed wire.

7. The ultrasonic probe according to claim 6, wherein the flexible printed wire is arranged to be folded approximately vertically to the element substrate.

8. The ultrasonic probe according to claim 5, comprising:
a circuit board connected to the electric connection unit to face a rear side of a surface of the element substrate on which the element is held.

9. The ultrasonic probe according to claim 8, wherein the circuit board is arranged approximately vertically to the element substrate.

10. The ultrasonic probe according to claim 1, wherein the center of the element is arranged to be offset from the center of the chassis in a range from 300 μm to 5 mm.

11. The ultrasonic probe according to claim 10, wherein the center of the element is arranged to be offset from the center of the chassis in a range from 500 μm to 4 mm.

12. The ultrasonic probe according to claim 11, wherein the center of the element is arranged to be offset from the center of the chassis in a range from 700 μm to 2 mm.

13. The ultrasonic probe according to claim 1, wherein the chassis is formed in a columnar shape.

14. The ultrasonic probe according to claim 13, wherein the chassis is formed in a circular columnar shape, and a diameter of the circular columnar chassis is in a range from 5 mm to 15 mm.

15. The ultrasonic probe according to claim 14, wherein the diameter of the circular columnar chassis is in a range from 6 mm to 12 mm.

16. The ultrasonic probe according to claim 15, wherein the diameter of the circular columnar chassis is in a range from 7 mm to 10 mm.

17. The ultrasonic probe according to claim 13, wherein the chassis is formed in a rectangular columnar shape.

18. An ultrasonic unit comprising:
a supporting member configured to arrange and support a plurality of ultrasonic probes each of which is the ultrasonic probe according to claim 1.

19. The ultrasonic unit according to claim 18, wherein the supporting member includes a recess and is formed in a shape of a hemisphere.

20. The ultrasonic unit according to claim 19, wherein a radius of the hemisphere is in a range from 100 mm to 150 mm.

21. The ultrasonic unit according to claim 20, wherein the radius of the hemisphere is in a range from 110 mm to 130 mm.

22. The ultrasonic unit according to claim 18, wherein the supporting member includes a light irradiation unit configured to irradiate a subject with light.

23. A subject information acquisition apparatus comprising:
the ultrasonic unit according to claim 18;
a light source unit; and
a signal processing unit,
wherein an acoustic wave from a subject is received to acquire information about the subject.

24. A subject information acquisition apparatus comprising:
the ultrasonic unit according to claim 18; and
a signal processing unit,
wherein an ultrasonic wave transmitted from the ultrasonic probe is emitted to a subject, an acoustic wave from the subject is received, to acquire information about the subject.
